(19) United States
(12) Patent Application Publication
(10) Pub. No.: US 2020/0060621 A1
(43) Pub. Date: Feb. 27, 2020

(54) IMPLANTABLE SYSTEM

(71) Applicants: UNIVERSITE GRENOBLE ALPES, SAINT MARTIN D'HERES (FR); CENTRE HOSPITALIER UNIVERSITAIRE GRENOBLE ALPES, LA TRONCHE (FR)

(72) Inventors: Philippe CINQUIN, SAINT NAZAIRE LES EYMES (FR); François Regis Pierre BOUCHER, GRENOBLE (FR); Pascal DEFAYE, GRENOBLE (FR); Pierre-Yves GUMERY, GRENOBLE (FR); Patrick TUVIGNON, ALBI (FR)

(21) Appl. No.: 16/466,062

(22) PCT Filed: Dec. 7, 2017
(86) PCT No.: PCT/EP2017/081877

§ 371 Date: Jun. 3, 2019

(30) Foreign Application Priority Data
Dec. 7, 2016 (FR) 1662059

(51) Int. Cl. A61B 5/00; A61B 5/08; A61B 5/0205

(52) U.S. Cl. CPC A61B 5/6871; A61B 5/02055; A61B 5/4094; A61B 5/0826

(57) ABSTRACT

The invention relates to an implantable system (10) comprising a first device (20), called a centralization device, which is able to be fixed in a fixation position to a wall of the stomach of a patient, the centralization device (20) being received in the stomach when the centralization device (20) is in the fixation position, and at least one second device (25). The implantable system (10) is characterized in that the centralization device (20) comprises a controller (45), an electrical supply (55) and an emitter/receiver (60) to permit communication between the centralization device (20) and the second device (25) when the second device (25) is in a functioning position, the second device (25) being situated outside the body of the patient when the second device (25) is in the functioning position.

The present invention relates to an implantable system.

A large number of implantable devices are used to monitor or stimulate certain organs of the human body. For example, cardiac stimulation devices (known as pacemakers) are implanted in numerous patients. These devices usually comprise an energy source such as a battery, one or more sensors enabling the behavior of the monitored organ to be monitored and/or a stimulation module provided to exert an action on the stimulated organ.

However, the batteries of these implanted devices need to be regularly recharged or replaced. In particular, in many cases, this replacement is performed by a surgical operation. Such a procedure is relatively expensive and inconvenient for the patient as it takes place in a hospital operating theater and an anesthetic is necessary, as well as a prolonged stay in hospital for postoperative monitoring. Furthermore, as with any surgical intervention, there are risks that the patient might contract an infection during the operation.

In other cases, implanted devices of the above-mentioned type are powered from outside by an energy storage module that is carried by the patient on the outside of his body. For example, there are some power devices that transmit the energy by ultrasound waves to the stimulation device, through the patient's skin and ribcage. However, ultrasound waves pass poorly through bone and great precision is therefore required when siting the ultrasound source, if the implanted device is located in the ribcage, to ensure a good supply to the implanted device. Furthermore, such a power device outside the patient's body is unsightly.

Some implanted devices may also be fitted with wired connectors, enabling an electrical connection or a transfer of fluid between the implantable device and an external device. In this way an electrical supply current or data measured by the sensors of the implanted device are exchanged with the external device. Here again, these connectors passing through the patient's skin are unsightly and necessarily present health risks as well as severe constraints for the patient in his daily life.

A need therefore exists for an implantable system that is less constraining for the patient.

To this end, an implantable system is proposed comprising:
a first device, called a centralization device, which is able to be fixed in a fixation position to a wall of the stomach of a patient, the centralization device being received in the stomach when the centralization device is in the fixation position, and
at least one second device,

the implantable system being characterized in that the centralization device comprises a controller, an electrical supply and an emitter/receiver to permit communication between the centralization device and the second device when the second device is in a functioning position, the second device being situated outside the body of the patient when the second device is in the functioning position.

According to various embodiments, the implantable system comprises one or more of the following characteristics, taken in isolation or according to all of the technically possible combinations:
the centralization device is configured to communicate with each second device by radiofrequency communication;
the centralization device comprises a sensor able to measure a value of a physiological parameter of the patient, the emitter/receiver being configured so as to transmit to a second device the measured values, the second device considered to be able to detect a physiological phenomenon of the patient on the basis of at least one of the measured values;
the implantable system comprises two second devices, and one of the second devices comprises at least one sensor able to measure a value of a physiological parameter of the patient, the emitter/receiver being configured to receive from the second device comprising a sensor the measured values and to transmit the measured values to the other second device, the other second device being able to detect a physiological phenomenon of the patient on the basis of at least one of the values received from the emitter/receiver;
the implantable system comprises a memory able to store the measured values for a period of more than or equal to one day;
the physiological phenomenon is a cardiac pathology;
the cardiac pathology is heart failure;
the sensor is an ultrasound emitter/receiver;
the sensor is an accelerometer able to measure values of an acceleration of the centralization device and the second device is configured to calculate a physiological parameter of the heart of the patient from the measured acceleration values;
the sensor is able to measure a difference in electrical potential, an acceleration, a noise, an orientation, a pH or a temperature;
the centralization device comprises a catheter able to convey a body fluid of the patient to the sensor, the sensor being able to measure a level of a biological marker in the body fluid;
the sensor comprises at least one light source and at least one detector of light radiation, the light source being configured to illuminate at least one portion of an organ of the patient with light radiation, the detector being configured to measure a value of a level of reflection of the light radiation, the controller being configured to calculate an oxygenation level of the blood circulating in the organ illuminated by the light source, based on the level of reflection;
the system comprises a plurality of sensors, and the second device is able to detect the physiological phenomenon on the basis of the values of at least two sensors;
the physiological phenomenon is chosen from the group formed by: cardiac dysrhythmia, atrial fibrillation, syncope, heart failure, sleep apnea, chronic obstructive pulmonary disease, emphysema, epilepsy, a swallowing disorder, an eating disorder;
when the centralization device is in the fixation position, the centralization device is in the upper part of the stomach;
the electrical supply comprises a removable reserve of electrical energy and a connector able to receive the reserve of electrical energy, the reserve of electrical energy being able to electrically supply the controller when the reserve of electrical energy is connected electrically to the connector in a connection position and preferably being configured to be swallowed by the patient and to move spontaneously to the connection position from a disconnection position in which the reserve of electrical energy is received in the stomach of the patient and is disconnected from the connector;
the electrical supply comprises an electrical energy generator able to generate an electrical current by reaction of at least one chemical species present in the body of the patient, particularly glucose;
the electrical supply comprises an electrical energy generator able to generate an electrical current by the conversion of mechanical energy into electrical energy.

Further features and advantages of the invention will emerge from the following description, given purely by way of non-limiting example and made with reference to the accompanying drawings, in which:

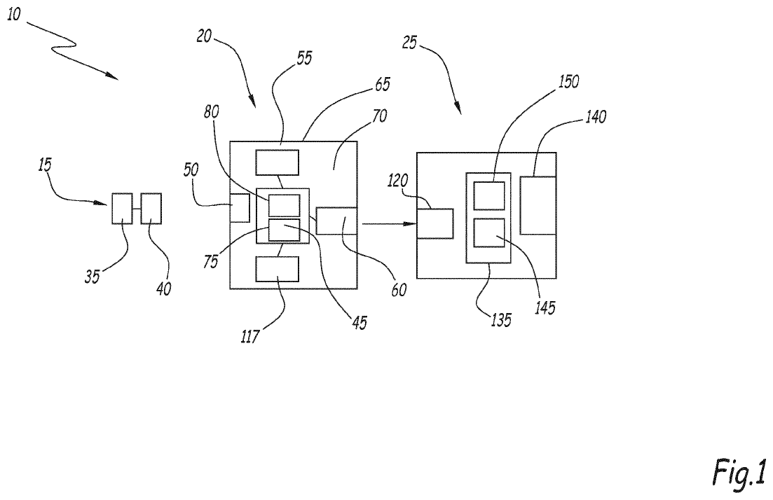

FIG. 1 is a diagram of an example of an implantable system comprising an electrical supply,

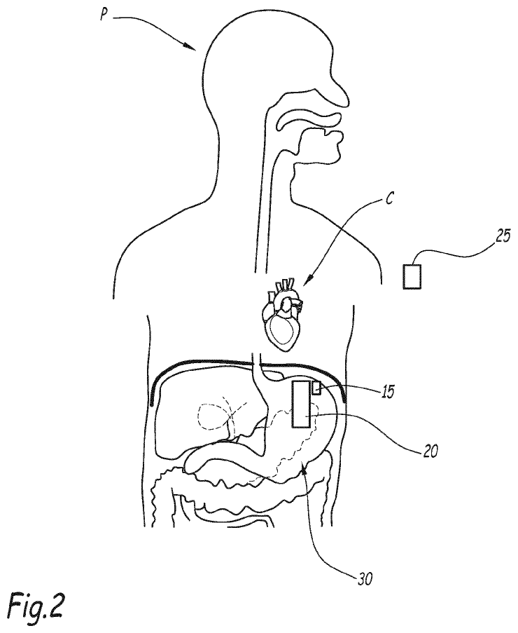

FIG. 2 is a schematic representation of the implantable device in FIG. 1, implanted in the body of a patient, and

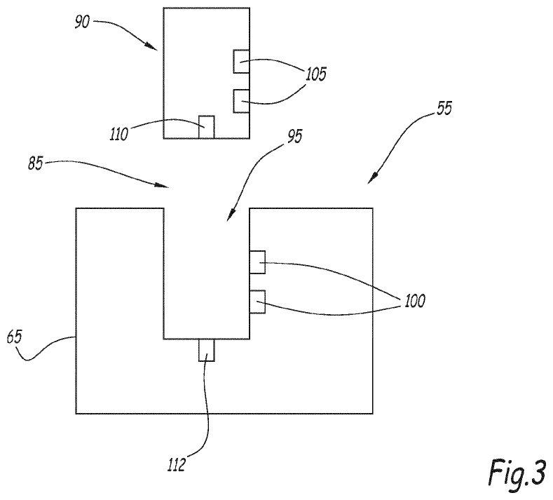

FIG. 3 is a schematic representation of the supply in FIG. 1.

A first example of an implantable system 10 is shown in FIG. 1.

The implantable system 10 comprises an anchor 15, a first device 20, called a centralization device, and at least one second device 25.

An “implantable system” means that at least one element among the list formed by the anchor 15 of the first device 20 and by the second device 25 is provided to be implanted in the human body.

In particular, “implantable” means that at least one element among the anchor 15, the centralization device 20 and the second device 25 is provided to remain in the body of a patient P for a period of strictly more than one week, preferably strictly more than one month, preferably longer than or equal to one year.

The implantable system 10 is shown schematically in FIG. 2 when the implantable system 10 is implanted in the body of the patient P.

The implantable system 10 is configured to detect at least one physiological phenomenon occurring in the patient P. A “physiological phenomenon” means a phenomenon concerning a function of an organ of the body of the patient P. The physiological phenomenon is a cardiac pathology. For example, the physiological phenomenon is a cardiac dysrhythmia. For example, the physiological phenomenon is an atrial fibrillation of the heart of the patient P.

As a variation, the physiological phenomenon is a syncope. According to another variation, the physiological phenomenon is a bradycardia.

According to another variation, the physiological phenomenon is a failure of the heart of the patient P.

According to the example in FIG. 2, the anchor 15 and the centralization device 20 are each implanted in the body of the patient P. The second device 25 is shown in a functioning position in FIG. 2.

The anchor 15 is able to be fixed in a predetermined position in the stomach 30 of the patient P.

For example, the anchor 15 is configured to be fixed in the top part of the stomach 30. In particular, the anchor 15 is configured to be fixed in the gastric fundus of the stomach 30. For example, the anchor 15 is provided to be fixed as closely as possible to the Angle of His in the gastric fundus.

As a variation, the anchor 15 is configured to be fixed in the lower part of the stomach 30.

The anchor 15 is configured to support the centralization device 20, preferably in a removable manner. In particular, the anchor 15 and the centralization device 20 are configured to be fixed to one another by a fixing device, and the anchor 15 is configured to hold the centralization device 20 in a fixation position when the anchor 15 is fixed in the stomach 30.

The anchor 15 comprises a head 35 and a first connector 40.

The head 35 is configured to anchor the anchor 15 in the predetermined position. In particular, the head 35 is configured to anchor the anchor 15 to the wall of the stomach 30.

The head 35 is, for example, a gastro-intestinal clip configured to grip between two branches of the head 35 a portion of the wall of the stomach 30.

As a variation, the head 35 is able to be sutured by a thread to the wall of the stomach.

According to another variation, the head 35 is able to be buried inside the gastric mucosa after the latter has been dissected.

The first connector 40 is configured to fix the centralization device 20 to the head 35.

The centralization device 20 comprises at least one sensor 117, a first controller 45, a second connector 50, an electrical supply 55, a first emitter/receiver 60 and a case 65. For example, the centralization device 20 comprises two sensors 117.

Each sensor 117 is outside the first controller 45 but is able to communicate with the first controller 45.

Each sensor 117 is configured to measure the value of a physiological parameter of the patient P. The physiological parameter is, for example, a parameter of an organ C.

The organ C is distinct from the stomach of the patient P. For example, at least one sensor 117 is able to measure values of a parameter of the heart.

For example, a sensor 117 is able to measure a value of an acceleration of the centralization device 20, such as an acceleration caused by a contraction of the heart C.

As a variation or in addition, a sensor 117 is able to measure a value of a difference in electrical potential between two electrodes of the sensor 117. The difference in electrical potential is, for example, measured between two points of the stomach wall, meaning that the two electrodes are in contact with the stomach wall. As a variation, the sensor 117 comprises only one electrode and is able to measure the difference in electrical potential between the electrode and the anchor 15. As a variation, the anchor 15 comprises two electrodes and the sensor 117 is able to measure a difference in potential between the two electrodes of the anchor 15.

The first controller 45 is a data processing unit. The first controller 45 comprises a first memory 75 and a first processor 80.

The first memory 75 is able to store the values measured by the sensor 117 for a storage period of longer than or equal to one hour, preferably longer than or equal to one day, preferably longer than or equal to one week.

The first processor 80 is able to handle and/or convert the data represented as electronic or physical quantities in the first memory 75 into other similar data corresponding to physical data in the first memory 75, in logs or other types of display, transmission or storage devices.

The first processor 80 is also configured to exchange information with the first emitter/receiver 60.

The second connector 50 is configured to cooperate with the first connector 40 to hold the centralization device 20 in the fixation position.

For example, the second connector 50 is configured to cooperate with the first connector 40 by clicking into it.

As a variation, the second connector 50 comprises a magnet configured to fix the second connector to the first connector. The magnet is, for example, an electromagnet.

According to another variation, the first connector 40 is configured to be connected to the second connector 50 by screwing. As a variation, the first connector 40 comprises one or preferably two bayonets complementary to fixation orifices made in the second connector 50.

Preferably, the second connector 50 is provided so that the centralization device 20 can be separated from the anchor 15. In particular, the second connector 50 is configured so that the centralization device 20 can be separated from the anchor 15 when the anchor 15 is fixed in the stomach 30 of the patient P.

The electrical supply 55 is shown in FIG. 3.

The electrical supply 55 is configured to supply the first controller 45 with a supply current C.

The electrical supply 55 comprises a third connector 85 and a first reserve of electrical energy 90.

The third connector 85 is configured to receive from the first reserve of electrical energy 90 the supply current C and to supply the first controller 45 with the first supply current C.

The third connector 85 is configured to receive the first reserve of electrical energy 90. In particular, the third connector 85 defines a cavity 95 configured to receive at least partially the first reserve of electrical energy 90 in a connection position.

According to the example in FIG. 3, the cavity 95 emerges on the outside of the case 65. In particular, the cavity 95 is configured to permit the insertion of the first reserve of electrical energy 90, from the outside of the case 65, into the cavity 95.

The third connector 85 also comprises two first electrical contacts 100, configured to be connected electrically to the first reserve of electrical energy 90 when the first reserve of electrical energy 90 is in the connection position. In particular, the two first electrical contacts 100 emerge on the inside of the cavity 95.

The first reserve of electrical energy 90 is configured to store electrical energy. In particular, the first reserve of electrical energy 90 is configured to be charged with electrical energy from outside of the body of the patient P and to discharge when the first reserve of electrical energy 90 is in the connection position. For example, the first reserve of electrical energy 90 comprises a battery. As a variation, the first reserve of electrical energy 90 comprises at least one capacitor or one supercapacitor.

The first reserve of electrical energy is configured to supply the first controller 45 with the supply current C when the first reserve of electrical energy 90 is in the connection position.

According to the example in FIG. 3, the first reserve of electrical energy 90 comprises two first electrical contacts 105 complementary to the first electrical contacts 100.

The first reserve of electrical energy 90 can be provided to be swallowed by the patient P.

According to a variation, the first reserve of energy 90 is able to be replaced by endoscopy.

In particular, the first reserve of electrical energy 90 has a volume strictly less than 6 milliliters (ml).

The first reserve of electrical energy 90 also has three dimensions, each measured in a respective direction, each direction being perpendicular to the other two directions, and each dimension is strictly less than 5 centimeters (cm).

The first reserve of electrical energy 90 is movable between the connection position and a disconnection position. When the first reserve of electrical energy 90 is in the disconnection position, the first reserve of electrical energy 90 is received into the stomach 30 of the patient P but is not electrically connected to the third connector 85. For example, when the first reserve of electrical energy 90 is in the disconnection position, the first reserve of electrical energy is totally extracted from the cavity 95.

The first reserve of electrical energy 90 is configured to move spontaneously from the disconnection position to the connection position. For example, the first reserve of electrical energy 90 comprises attractors 110.

Preferably, the first reserve of electrical energy 90 is configured to eject from the third connector 85 another spent first reserve of electrical energy 90, if any. In other words, the first reserve of electrical energy 90 is configured so as to cause, if a spent first reserve of electrical energy 90 is in the connection position, the disconnection of the spent first reserve of electrical energy 90 and the movement of the spent first reserve of electrical energy from the connection position to the disconnection position.

The attractors 110 are configured so as to exert on the first reserve of electrical energy 90, when the first reserve of electrical energy 90 is in the disconnection position, a force tending to move the first reserve of electrical energy 90 from the disconnection position to the connection position.

Furthermore, the attractors 110 are configured to hold the first reserve of electrical energy 90 in the connection position.

The attractors 110 comprise, for example, a first magnet able to cooperate with a second magnet 112 of the third connector 85. As a variation, the first magnet is able to cooperate with a ferromagnetic portion of the third connector 85. The first magnet and the second magnet 112 are, for example, electromagnets.

The first emitter/receiver 60 is configured to exchange information with the second device 25 when the second device 25 is in the operating position. The first emitter/receiver 60 thus forms means of communication with the second device 25.

The first emitter/receiver 60 is, for example, a radiofrequency communication module. “Radiofrequency communication module” means that the first emitter/receiver 60 is configured to communicate with the second device 25 via a signal comprising at least one radiofrequency electromagnetic wave. Radiofrequency electromagnetic waves are electromagnetic waves having a frequency of between 3 kilohertz and 3 gigahertz.

According to one embodiment, the first emitter/receiver 60 is able to exchange information with the second device 25 according to a Bluetooth Low Energy protocol. The Bluetooth Low Energy protocol is a protocol based on a “Bluetooth special interest group” standard and operating within the range of between 2400 megahertz (MHz) and 2483.5 MHz.

As a variation, methods for the transmission of information within the ranges of 402-405 megahertz (MHz) (Medical Implant Communication Service) or 2360-2390 MHz (Medical Body Area Networks) can be used.

The case 65 is configured to isolate the first controller 45 from the outside of the case 65. For example, the case 65 defines a chamber receiving at least the first controller 45 and the first emitter/receiver 60.

The second device 25 is not implanted inside the body of the patient P when the device 25 is in the functioning position. In particular, the second device 25 is located outside the body of the patient P when the second device 25 is in the functioning position.

For example, the second device 25 is installed in a doctor's practice. As a variation, the second device 25 is installed at the home of the patient P. As a variation, the second device 25 is carried by the patient, for example the second device is a mobile telephone such as a smartphone, a tablet, a dedicated device or even a module integrated into a mobile telephone or tablet.

The second device 25 is a display device able to transmit information to a user U.

The second device 25 is also configured to detect the physiological phenomenon on the basis of the measured values.

The second device 25 comprises a second emitter/receiver 120, a second controller 135 and a man/machine interface 140.

The second emitter/receiver 120 is configured to exchange information with the first emitter/receiver 60.

The second controller 135 is configured to detect the physiological phenomenon on the basis of the values measured by the sensor 117.

The second controller 135 comprises a second memory 145 and a second processor 150.

The man/machine interface comprises, for example, a screen and loudspeaker.

A method for monitoring the organ C is implemented by the implantable system 10.

The monitoring method comprises an implantation step, a measurement step, a transfer step, a detection step and a signaling step.

During the implantation step, the anchor 15 and the centralization device 20 are implanted in the body of the patient P. The second device 25 is not implanted in the body of the patient P.

During the measurement step, each sensor 117 measures the values of the parameter. For example, the sensor 117 periodically measures the values of the corresponding parameter for a predetermined period of time.

The measured values are stored in the first memory 75.

The measured values are stored in the first memory 75 for a period longer than or equal to one hour, for example longer than or equal to one day, for example longer than or equal to one week, for example longer than or equal to one month.

During the transfer step, the measured values are transferred, by the centralization device 20, to the second device 25. For example, the transfer step is implemented when the patient P visits his assigned doctor's practice.

As a variation, the transfer step is implemented periodically when the patient P is at home, for example once a day. According to a variation, when the patient carries the second device 25 on his person, for example when the second device 25 is integrated into a mobile telephone, the transfer step is implemented for a period of less than or equal to one hour.

For example, thanks to the measured values of difference in potential, the second device 25 displays an electrocardiogram of the patient P.

After each transfer step, the first memory 75 is erased. In particular, the values of stored parameters are erased.

During the detection step, the second controller 135 detects the physiological phenomenon on the basis of the measured values.

According to a variation, the second controller 135 calculates the operating data on the basis of the measured values and detects the physiological phenomenon on the basis of the operating data. The operating data are data derived from the measured values. For example, a heart rate is an example of operating data calculated on the basis of measurements of a difference in potential between two electrodes or on the basis of acceleration values of the centralization device 20. An interval of time between two waves of polarization of an atrium of the heart is another example of operating data, and a variation of this period of time is another example of operating data. A correlation coefficient obtained by an adjustment of a set of measured values with a set of reference values or a reference function is another example of operating data.

For example, the second controller 135 compares each measured value or each value of operating data to a predetermined threshold and detects the physiological phenomenon if one of the measured values exceeds or is equal to the predetermined threshold.

According to another example, the physiological phenomenon is detected if several measured values or several values of operating data exceed or are equal to a predetermined threshold for a predetermined period of time. According to a variation, the second controller 135 detects the physiological phenomenon if one or more of the measured values or values of operating data exceeds or is equal to a predetermined threshold, or even if one or more of the measured values or values of operating data is not included within a range of data framed by two predetermined thresholds.

According to another example, the physiological phenomenon is detected if several measured values or several values of operating data belong to a predetermined region of the space Rⁿ, where R is the set of real values and n represents the number of values. According to a variation, the second controller 135 detects the physiological phenomenon if one or more of the measured values or values of operating data belong to a predetermined region of the space Rⁿ.

The predetermined thresholds or the predetermined region of the space Rⁿ are, for example, determined by an automatic learning method. The automatic learning methods are also called “machine learning”. For example, the implantable system records the measured values while the patient undergoes more conventional examinations (external electrocardiogram or morphological examinations, for example ultrasound examinations, possibly performed during operating tests, for example stress tests). These more conventional examinations are chosen to permit a diagnosis to be made, and a machine learning method permits combinations of the values measured by the implantable system to be determined that correspond to the physiological or pathological phenomenon studied. In everyday life, when the measured values confirm these characteristics, the second controller 135 will detect the physiological or pathological phenomenon.

According to one variation, the second controller 135 detects the physiological phenomenon on the basis of values measured by at least two separate sensors 117.

According to one embodiment, the second controller 135 detects atrial fibrillation on the basis of acceleration values and/or measured values of electrical voltage. The second controller 135 detects, for example, atrial fibrillation on the basis of an analysis of electrical voltage values to detect the presence or absence, in the electrocardiogram, of a representative signal of a wave P of polarization of the heart's atrium. As a variation, the analysis of the variation of the interval of time between two waves R enables atrial fibrillation to be detected.

The signaling step is then implemented.

During the signaling step, the second controller 135 commands the emission, through the man/machine interface 140, of a warning signal for the patient P and/or his doctor.

The warning signal informs the patient P and/or his doctor that the physiological phenomenon has occurred. For example, the warning signal contains a date on which the physiological phenomenon occurred, a frequency of appearance of the physiological phenomenon, a duration of the physiological phenomenon, or even a heart rate of the patient during the physiological phenomenon.

The warning signal is, for example, simultaneously transmitted to an emergency assistance agency. For example, the warning signal is sent to an emergency assistance agency via a wireless telephone network. As a variation, the warning signal is sent via the internet.

As the centralization device 20 is in the stomach, the replacement of the first reserve of electrical energy 90 is easy and can, for example, be simply and quickly performed by an endoscopy through the esophagus. Furthermore, the replacement of the first reserve of electrical energy 90 poses few risks of infection as no incision is made.

The use of attractors 110 makes the installation of the first reserve of electrical energy 90 even simpler, even without an endoscopy, as the patient P need only swallow the first reserve of electrical energy 90.

Furthermore, the implantable system 10 does not involve the patient P permanently carrying the means of electrical energy storage outside his body, or unsightly electrical conductors protruding from the body of the patient P. The implantable system 10 therefore imposes few constraints on the patient.

The upper part of the stomach 30 is close to the heart C, and the contractions of the heart C therefore cause the centralization device 20 to move. Since an acceleration sensor 117 is positioned in the stomach, atrial fibrillation is effectively detected, not only by an electrical measurement of the heart's activity but also by measuring the acceleration of the centralization device 20.

The implantable system thus improves the safety of the patient P.

Furthermore, positioning the centralization device 20 in the stomach also reduces the constraints for the patient P.

Furthermore, the data acquired by the sensors 117 is processed by the second device 25, which is located outside the patient's body. The electrical consumption of the centralization device 20 is thus limited, which reduces the frequency with which the electrical supply of the centralization device 20 must be changed. The constraints for the patient P are thus further limited.

According to a variation of the first example, the measurement step is implemented during non-continuous time ranges, separated by second time ranges during which no value is measured. According to one embodiment, the first time ranges last for 30 seconds and the second time ranges last for 30 minutes.

Thus, the first device 20 is dormant for a large proportion of time. The energy consumption of the implantation system is therefore reduced. This embodiment is particularly suitable for the detection of physiological phenomena that have a slow development, such as heart failure.

The first example of an implantable system 10 has been given for the case of detecting atrial fibrillation in the patient P. As a variation, the implantable system 10 can also enable other cardio-vascular diseases to be detected, such as heart failure. However, it should be noted that positioning the centralization device in the stomach 30 of the patient P enables the parameters of a wide variety of organs C to be measured. The implantable system 10 can therefore be adapted to detect a large number of distinct physiological phenomena.

For example, the physiological phenomenon is a respiratory disorder. Chronic obstructive pulmonary disease is an example of a respiratory disorder.

As a variation, the physiological phenomenon is emphysema.

According to another variation, the physiological phenomenon is epilepsy.

According to another variation, the physiological phenomenon is an eating disorder.

According to another variation, the physiological phenomenon is sleep apnea. For example, the sensor 117 is able to detect a contraction of the diaphragm and the second controller 135 is configured to detect sleep apnea if a period of time without contraction of the diaphragm lasts longer than or equal to a predetermined threshold.

According to another variation, the physiological phenomenon is a swallowing disorder, or even an eating disorder such as insufficient hydration.

Moreover, numerous types of sensors 117 can be used.

For example, the sensor 117 is a light emitter/receiver comprising at least one light source and at least one detector of light radiation. For example, the sensor 117 comprises two light sources. According to one embodiment, the sensor 117 then comprises two detectors of light radiation.

The light source(s) are configured to illuminate at least one portion of an organ of a patient P with light radiation. For example, each light source is configured to illuminate a portion of the heart muscle. The portion is, for example, a portion of a heart cavity.

The illuminated organ is, as a variation, a blood vessel such as the aorta of the patient P.

As a variation, the blood vessel is the vena cava.

According to another variation, the illuminated organ is the gastric wall.

According to another variation, the illuminated organ is the diaphragm.

Each light radiation has a wavelength. For example, at least one wavelength is chosen for its level of absorption or reflection by certain molecules. For example, at least one light radiation has a visible wavelength. A wavelength equal to 660 nanometers is an example of visible wavelength.

According to one variation, the light radiation of at least one light source is an infrared light radiation. For example, the infrared radiation has a wavelength equal to 950 nanometers.

According to one embodiment, one wavelength is equal to 660 nanometers and another wavelength is equal to 950 nanometers. These wavelengths are absorbed and/or reflected differently by the unsaturated hemoglobin (known as Hb) and saturated hemoglobin (known as HbO₂), and thus enable a good evaluation of the relationship between these two molecules.

The detector is configured to measure a value of a level of reflection of each light radiation on the illuminated organ. For example, the detector comprises a photodiode.

As a variation, the detector is configured to measure a level of absorption of each light radiation.

The second controller 135 is configured to receive from the detector the values of level of reflection measured, and to calculate a level of oxygenation of the blood circulating in the illuminated organ on the basis of the values received. For example, the second controller 135 is configured to calculate a level of oxygenation of the capillary blood circulating in the gastric wall. As a variation, the second controller 135 is configured to calculate a level of oxygenation of the capillary blood circulating in the heart muscle, or blood present in the cardiac cavities, or blood present in the aorta or in the vena cava.

The physiological phenomenon that the implantable system 10 is able to detect is therefore a drop in the oxygen saturation of the hemoglobin of the patient P. This drop is caused, for example, by a heart or respiratory disease.

According to another variation, at least one sensor is a sensor of a biological marker of a body fluid F of the patient P. The body fluid F is, for example, the contents of the stomach or the extracellular fluid at the stomach wall where the first device 20 is implanted.

According to another variation, the centralization device 20 comprises a first catheter configured to convey a body fluid F of the patient P from the organ C to the sensor 117. For example, the organ C is the peritoneum and the fluid F is the peritoneal fluid.

The biological marker is, for example, glucose. As a variation the biological marker is an ion present in the body fluid F. For example, the sensor 117 is capable of measuring the pH of the body fluid F. In particular, the sensor 117 is capable of measuring the pH of the gastric contents or peritoneal fluid of the patient P.

The sensor 117 is therefore capable of measuring a level of biological marker in the body fluid F. For example, the sensor 117 is capable of measuring a glucose level in the peritoneal fluid, and the first calculator 45 is capable of estimating a blood sugar value of the patient P.

According to another variation, at least one sensor 117 is configured to estimate an orientation of the centralization device 20 in relation to the vertical. The first controller 45 is then configured to detect a position of the patient P. In particular, the first controller 45 is configured to detect a lying position, a sitting position or an intermediate position between the lying and sitting positions of the patient P.

For example, the sensor 117 comprises at least one element from the list formed by: a gyroscope, a magnetometer and an accelerometer.

According to another variation, at least one sensor 117 is capable of measuring a body temperature of the patient P. According to another variation, at least one sensor 117 is a microphone.

The sensor 117 is configured to measure a noise emitted by the organ C. The second controller 135 is capable of detecting the physiological phenomenon on the basis of the analysis of the noises measured. For example, the second controller 135 is capable of detecting a heart rate disorder, a heart valve anomaly, a lung disease or even a digestive disorder on the basis of the noises measured.

According to another variation, at least one sensor 117 is an ultrasound emitter/receiver. For example, the sensor 117 is capable of emitting at least one ultrasonic beam and of measuring a parameter of a beam reflected on all or part of the heart of the patient P. The parameter of the beam is, for example, a level of reflection, or even a phase shift.

The second controller 135 is, for example, capable of calculating a dimension or an amplitude of contraction of the heart of the patient P on the basis of measurements provided by the sensor 117. In particular, the second controller 135 is configured to calculate an ejection fraction of the heart on the basis of the dimensions or amplitudes of contraction measured.

As a variation, the beam emitted is reflected on a blood vessel such as the aorta and the operating data comprise a variation in the diameter of the blood vessel. According to a variation, the vessel is the vena cava.

According to one embodiment, the sensor 117 is configured to evaluate a blood flow or a blood pressure in a blood vessel by Doppler effect.

A pressure sensor is another example of the sensor 117.

According to another variation, the first device 20 comprises several sensors 117, and the second controller 135 is configured to detect the physiological phenomenon on the basis of values measured by at least two sensors 117. According to another variation, the second controller 135 is capable of detecting at least two distinct physiological phenomena on the basis of values measured by the sensor(s) 117.

A second example of an implantable system 10 will now be described. The elements identical to the first example of an implantable system 10 in FIG. 1 will not be described again. Only the differences will be highlighted.

The implantable system 10 comprises two second devices 25.

One of the two devices 25 is a display device as previously described. The other second device 25 will henceforth be referred to by the expression “measurement device”. In order to distinguish one from the other, the two second devices 25 will henceforth be referred to in the second example by the expressions “display device” and “measurement device” respectively.

The measurement device 25 is implanted, in its operating position, in the body of the patient P. In particular, the measurement device 25 is implanted outside the stomach 30 of the patient P.

According to a variation, the measurement device 25 is not implanted in the body of the patient P but is carried by the patient P. For example, the measurement device 25 is fixed around a limb of the patient P by a strap. It will be noted that other operating positions and other methods of fixation can be envisaged.

The measurement device 25 does not comprise a man-machine interface 140.

The measurement device 25 comprises at least one sensor 117.

The second emitter/receiver 120 of the measurement device 25 is configured to transmit to the first emitter/receiver 60 the measured values.

The first memory 75 is, furthermore, configured to store the values measured by the sensor(s) of the measurement device 25.

The first emitter/receiver 60 is configured to transmit to the display device the values measured by the sensor(s) 117 of the measurement device 25.

The second controller 135 is then configured to detect at least one physiological phenomenon on the basis of at least one of the values measured by the sensor(s) 117 of the measurement device 25.

The implantable system 10 is then capable of being used for the detection of physiological phenomena for which positioning of the sensor 117 in the stomach is not favorable.

According to a variation of the second example, the centralization device 20 does not comprise a sensor 117. Only the measurement device 25 comprises at least one sensor 117. The centralization device 20 then plays a role of storage and transmission to the display device 25 of the measured values.

According to another variation, the implantable system 10 comprises at least two measurement devices 25.

A third example of an implantable system 10 will now be described. The elements identical to the first example of implantable system 10 will not be described again. Only the differences will be highlighted.

The electrical supply 55 does not comprise a third connector 85 or an electrical energy reserve 90.

The electrical supply 55 comprises an electrical energy generator. An “electrical energy generator” means that the electrical energy generator is not configured to be charged with electrical energy by an electrical current.

The electrical energy generator is capable of generating at least one electrical current by reaction of at least one chemical species present in the body of the patient P. More precisely, the electrical energy generator is capable of generating the supply current C.

For example, the electrical energy generator comprises two electrodes, the electrodes being bathed in the gastric juices of the patient P when the centralization device 20 is in the fixation position. As a variation, the electrodes of the electrical energy generator are provided to be bathed in the intestine of the patient P when the centralization device 20 is in the fixation position.

Each electrode comprises at least one enzyme. As a variation, each electrode comprises at least one microorganism. For example, each electrode of the electrical energy generator comprises an electrical conductor covered with the enzyme or microorganism, the assembly thus formed being surrounded by a membrane. The membrane is, for example, configured to be passed through by certain chemical species naturally present in the stomach or intestine of the patient P.

When the electrodes of the generator of electrical energy are immersed in the gastric juices or in the intestinal fluid, one of the electrodes plays the role of anode in a redox reaction involving a first chemical species. Simultaneously, the other electrode plays the role of a cathode in a redox reaction involving a second chemical species.

By oxidation and the simultaneous reduction of the first chemical species and of the second chemical species, an electrical voltage appears between the two electrical conductors. The supply current C is then generated.

The first chemical species is, for example, glucose. The second chemical species is, for example, oxygen.

The third example of an implantable system 10 does not require an electrical energy reserve 90 to be electrically charged or the electrical energy reserve 90 to penetrate the body of the patient P.

Here, too, the constraints for the patient P are reduced.

According to a fourth example, the electrical energy generator is capable of generating at least one electrical current by conversion of a mechanical energy into an electrical energy. In particular, the electrical energy generator is capable of generating at least one electrical current on the basis of the movements of the stomach 30.

According to a fifth example, the first controller 45 detects the physiological phenomenon on the basis of the measured values. Only one message comprising the results of the detection is transmitted to the second device 25.

In the above description, the functions of the implantable system 10 have been separated into several examples in order to make them more easily understood by the reader. However, it will be noted that the preceding examples are capable of being combined in order to generate new embodiments.

The implantable system 10 is particularly suitable for the detection of heart failure. In this case, at least one of the sensors 117 is chosen from the assembly formed by: a sensor measuring a difference in electrical potential, an accelerometer, an ultrasound emitter/receiver, a light emitter/receiver and a microphone. For example, two sensors 117 integrated into the centralization device 20 are chosen from the assembly formed by: a sensor measuring a difference in electrical potential, an accelerometer, an ultrasound emitter/receiver, a light emitter/receiver and a microphone.

The second controller 135 is then configured to detect heart failure on the basis of values measured by the sensors 117 and/or on the basis of operating data calculated on the basis of values measured by the sensors 117.

For example, the second controller 135 is configured to detect heart failure on the basis of values of electrical potential.

For example, the second controller 135 is configured to detect heart failure on the basis of acceleration values.

According to one embodiment, the second controller 135 is configured to detect heart failure on the basis of a combination of values of electrical potential, acceleration and orientation of the patient.

For example, the second controller 135 if configured to detect heart failure on the basis of oxygenation level values of the blood or an ejection fraction of the heart.

For example, the second controller 135 is configured to detect heart failure on the basis of a combination of values measured by all of the sensors 117 and of values of all of the operating data.

If one of the sensors 117 is an ultrasound emitter/receiver, the second controller 135 calculates an ejection fraction of the heart on the basis of variations in the dimensions of the heart and detects heart failure if the ejection fraction is below or equal to a corresponding threshold.

As a variation, heart failure is detected on the basis of an analysis of the noises made by the heart or lung.

Moreover, the above description has been given in the case where the anchor 15 and the centralization device 20 form two separate devices. It will be noted that the centralization device 20 and the anchor are capable of forming a single device, the anchor 15 and the centralization device 20 not then being separable from one another. For example, the anchor 15 is formed in one piece with the case 65 of the centralization device 20.

According to yet another example, the head 35 comprises at least one pad located outside the stomach 30. For example, the head 35 comprises two pads.

Each pad is configured to rest against the outer face of the wall of the stomach 30 and to be connected to the implantable device 20 so as to exert a force tending to hold the implantable device 20 flat against the inner face of the wall of the stomach 30. According to a variation, each pad is configured to be placed between the visceral sheet and the parietal sheet of the peritoneum and to rest against the visceral sheet in order to hold the implantable device 20 flat against the inner face of the wall of the stomach 30.

Each pad is, for example, a plate. As a variation, each pad comprises a wire mesh stretched over a frame, in particular a flexible frame capable of being folded and inserted in an endoscope or a hollow needle.

The first connector 40 comprises, for example, one or more rings integral with the case 65. Each pad is, for example, fixed to the implantable device 20 by one or more wires fixed to one or more of the rings.

The fixation by one or more pads allows the pressure exerted by the implantable device to be distributed over a larger surface area of the wall of the stomach 30 and so reduce the pressure thus exerted. Furthermore, this method of fixation does not entail generating in the stomach wall a fold that reduces the volume of the stomach, which is capable of generating tensions in the anchor that is fixed therein. Since the forces exerted on the stomach wall are reduced, the risks of appearance of an inflammatory reaction of the gastric mucosa are limited.

1. Implantable system (10) comprising:
a first device (20), called a centralization device, which is able to be fixed in a fixation position to a wall of the stomach (30) of a patient (P), the centralization device (20) being received in the stomach (30) when the centralization device (20) is in the fixation position, and
at least one second device (25),
the implantable system (10) being characterized in that the centralization device (20) comprises a controller (45), an electrical supply (55) and an emitter/receiver (60) to permit communication between the centralization device (20) and the second device (25) when the second device (25) is in a functioning position, the second device (25) being situated outside the body of the patient (P) when the second device (25) is in the functioning position, wherein:
the centralization device (20) comprises a sensor (117) able to measure a value of a physiological parameter of the patient (P), the emitter/receiver (60) being configured so as to transmit to a second device (25) the measured values, the second device (25) considered to be able to detect a physiological phenomenon of the patient (P) on the basis of at least one of the measured values, or
the implantable system (10) comprises two second devices wherein one of the second devices (25) comprises at least one sensor (117) able to measure a value of a physiological parameter of the patient (P), the emitter/receiver (60) being configured to receive from the second device (25) comprising a sensor (117) the measured values and to transmit the measured values to the other second device, the other second device (25) being able to detect a physiological phenomenon of the patient (P) on the basis of at least one of the values received from the emitter/receiver (60).

2. Implantable system (10) according to claim 1, wherein the centralization device (20) is configured to communicate with each second device (25) by radiofrequency communication.

3. Implantable system (10) according to claim 1, wherein the controller (45) comprises a memory (75) able to store the measured values for a period of more than or equal to one day.

4. Implantable system (10) according to claim 1, wherein the physiological phenomenon is a cardiac pathology.

5. Implantable system (10) according to claim 4, wherein the cardiac pathology is heart failure.

6. Implantable system (10) according to claim 1, wherein the sensor (117) is an ultrasound emitter/receiver.

7. Implantable system (10) according to claim 1, wherein the sensor (117) is an accelerometer able to measure values of an acceleration of the centralization device (20) and the second device (25) is configured to calculate a physiological parameter of the heart of the patient (P) from the measured acceleration values.

8. Implantable system (10) according to claim 1, wherein the sensor (117) is able to measure a difference in electrical potential, an acceleration, a noise, an orientation, a pH or a temperature.

9. Implantable system (10) according to claim 1, wherein the centralization device (20) comprises a catheter able to convey a body fluid (F) of the patient (P) to the sensor (117), the sensor (117) being able to measure a level of a biological marker in the body fluid (F).

10. Implantable system (10) according to claim 1, wherein the sensor (117) comprises at least one light source and at least one detector of light radiation, the light source being configured to illuminate at least one portion of an organ of the patient (P) with light radiation, the detector being configured to measure a value of a level of reflection of the light radiation, the controller (45) being configured to calculate an oxygenation level of the blood circulating in the organ illuminated by the light source, based on the level of reflection.

11. Implantable system (10) according to claim 1, comprising a plurality of sensors (117), wherein the second device (25) is able to detect the physiological phenomenon on the basis of the values of at least two sensors (117).

12. Implantable system (10) according to claim 1, wherein the physiological phenomenon is chosen from the group formed by: a cardiac dysrhythmia disorder, atrial fibrillation, syncope, heart failure, sleep apnea, chronic obstructive pulmonary disease, emphysema, epilepsy, a swallowing disorder, an eating disorder.

13. Implantable system (10) according to claim 1, wherein the electrical supply (55) comprises a removable reserve of electrical energy (90) and a connector (85) able to receive the reserve of electrical energy (90), the reserve of electrical energy (90) being able to electrically supply the controller (45) when the reserve of electrical energy (90) is connected electrically to the connector (85) in a connection position and preferably being configured to be swallowed by the patient (P) and to move spontaneously to the connection position from a disconnection position in which the reserve of electrical energy (90) is received in the stomach (30) of the patient (P) and is disconnected from the connector (85).

14. Implantable system (10) according to claim 1, wherein the electrical supply (55) comprises an electrical energy generator able to generate an electrical current by reaction of at least one chemical species present in the body of the patient (P), particularly glucose.

15. Implantable system (10) according to claim 1, wherein the electrical supply (55) comprises an electrical energy generator able to generate an electrical current by the conversion of mechanical energy into electrical energy.
